(19) United States
(12) Patent Application Publication
(10) Pub. No.: US 2023/0099837 A1
(43) Pub. Date: Mar. 30, 2023

(54) COMPOSITIONS AND METHODS FOR TREATING NEURODEGENERATIVE DISORDERS WITH RIPK1/RIPK3 INHIBITORS

(71) Applicant: Arizona Board of Regents on Behalf of the University of Arizona, Tucson, AZ (US)

(72) Inventors: May Khanna, Tucson, AZ (US); Jonathan Lares Sanchez, Tucson, AZ (US); Vijay Gokhale, Tucson, AZ (US)

(21) Appl. No.: 17/792,917

(22) PCT Filed: Jan. 22, 2021
(86) PCT No.: PCT/US21/14580

§ 371 Date: Jul. 14, 2022

Related U.S. Application Data
(60) Provisional application No. 62/965,634, filed on Jan. 24, 2020.
(60) Provisional application No. 63/032,182, filed on May 29, 2020.

(51) Int. Cl. A61K 31/437; A61K 31/4402; A61P 25/28

(52) U.S. Cl. CPC A61K 31/437; A61K 31/4402; A61P 25/28

(57) ABSTRACT

This invention relates generally to neurodegenerative diseases and conditions (e.g., Alzheimer's disease) characterized with aberrant RIPK1/RIPK3 binding, RIPK3/MLKL binding, necrosome formation, and/or necroptosis activation. This invention further relates to methods and compositions for treating such neurodegenerative diseases and conditions with pharmaceutical compositions comprising agents capable of inhibiting RIPK1/RIPK3 binding and/or RIPK3/MLKL binding.

### CROSS-REFERENCE TO RELATED APPLICATIONS

This application claims the priority benefit of U.S. Provisional Application No. 63/032,182, filed May 29, 2020 and U.S. Provisional Application No. 62/965,634, filed Jan. 24, 2020, which are incorporated herein by reference in their entireties.

### STATEMENT REGARDING FEDERALLY SPONSORED RESEARCH OR DEVELOPMENT

This invention was made with government support under Grant No. AG044402 awarded by National Institutes of Health. The government has certain rights in the invention.

### FIELD OF THE INVENTION

This invention relates generally to neurodegenerative diseases and conditions (e.g., Alzheimer's disease) characterized with aberrant RIPK1/RIPK3 binding, RIPK3/MLKL binding, necrosome formation, and/or necroptosis activation. This invention further relates to methods and compositions for treating such neurodegenerative diseases and conditions with pharmaceutical compositions comprising agents capable of inhibiting RIPK1/RIPK3 binding and/or RIPK3/MLKL binding.

### BACKGROUND OF THE INVENTION

There is an urgent need to develop novel therapies for neurodegenerative diseases and conditions such as Alzheimer's disease (AD). 10% of persons over age 65 and up to 50% over age 85 have dementia, with over 30 million people affected worldwide. AD affects over 26 million people worldwide and currently there is no cure for the disease. With the growing number of people living to older ages, there is an urgency to better understand elements of the pathogenic pathway, discover agents that target these elements, and establish their roles in the treatment and prevention of AD.

As such, improved methods for treating neurodegenerative disorders (e.g., AD) are needed.

The present invention addresses this need.

### SUMMARY

Activation of a programmed necrosis, termed necroptosis, is an essential form of cell death that is characterized by cellular swelling, plasma membrane disruption, and release of cellular contents known as damage associated molecular patterns (DAMPs) that produce an inflammatory response (see, Roh, J. S. & Sohn, D. H. Immune Network 18 (2018)). Necroptosis is initiated in response to inflammation triggers via death receptors in the TNF superfamily (see, Zhu, F., Zhang, W., Yang, T. & He, S. D. Journal of Zhejiang University-Science B 20, 399-413 (2019)). During necroptosis activation, three key proteins play a critical role in the resultant cell death: RIPK1, RIPK3, and MLKL.

Upon cellular death stimulus to TNF cellular death receptors, RIPK1 autophosphorylates and leads to binding and phosphorylation of RIPK3 through specific interactions between their N terminal kinase domains and C terminal RIP homotypic interaction motifs in an amyloid formation known as the necrosome (see, Mompean, M. et al. Cell 173, 1244-+(2018); Newton, K. Cell Biology 25, 347-353 (2015)). Upon RIPK3 activation, MLKL is subsequently recruited and phosphorylated, leading to MLKL conformational changes and oligomerization. Activated MLKL translocates to the cellular membrane and results in eventual cell death through membrane rupture via either direct pore formation or indirect methods associated with sodium and calcium ion channels (see, Sun, L. M. & Wang, X. D. Trends in Biochemical Sciences 39, 587-593 (2014); Chen, X. et al. Cell Research 24, 105-121 (2014); Cai, Z. Y. et al. Nature Cell Biology 16, 55-+(2014)).

There are a small number indications that necroptosis may play a role during the development of several organisms under certain conditions (see, Grootjans, S., Vanden Berghe, T. & Vandenabeele, P. Cell Death and Differentiation 24, 1184-1195 (2017)). However, necroptosis's main role in cell death appears to function as a means to remove damaged cells and as part of the immune response (see, Kaczmarek, A., Vandenabeele, P. & Krysko, D. V. Immunity 38, 209-223 (2013); Upton, J. W., Kaiser, W. J. & Mocarski, E. S. Cell Host & Microbe 26, 564-564 (2019); Nailwal, H. & Chan, F. K. M. Cell Death and Differentiation 26, 4-13 (2019)). Certain cell types, such as murine macrophages, preferentially undergo necroptosis under conditions of caspase inhibition and in response to double stranded RNA or lipopolysaccharides (see, He, S. D., Liang, Y. Q., Shao, F. & Wang, X. D. Proceedings of the National Academy of Sciences of the United States of America 108 20054-20059 (2011)). In addition, necroptosis provides an alternative approach to deal with pathogens that are able to circumvent apoptosis through expression of proteins that inhibit cellular caspases (see, Dondelinger, Y., et al., Trends in Cell Biology 26, 721-732 (2016)). Although activation of the innate immune system through necroptosis activation is beneficial, necroptosis has also been observed in several different diseases ranging from pulmonary, cardiovascular, and neurodegenerative diseases such as Multiple sclerosis (MS), Amyotrophic lateral sclerosis (ALS), Parkinson's disease (PD), and Alzheimer's disease (AD) (see, Choi, M. E., et al., Jci Insight 4 (2019)). Therefore, inhibition of necroptosis in these disease states represent a potential target for therapeutic intervention.

Towards this goal, the formation of the necrosome and subsequent MLKL activation in necroptosis has led to an increase in interest in the development of these signaling components. Indeed, several kinase inhibitors have been developed to prevent RIPK11 and/or RIPK3 activation through either a type I, II, or III kinase binding modes (see, Martens, S., et al., Trends in Pharmacological Sciences 41, 209-224 (2020)). RIPK11 kinase inhibitors have been demonstrated to ameliorate detrimental necroptotic cellular damage in ischemia reperfusion injury and atherosclerosis models of cardiovascular disease (see, Shi, Z. W., et al., Frontiers in Pharmacology 9 (2018)). Similarly, RIPK1 kinase inhibitors in pulmonary, hepatic, renal, and several neurodegenerative diseases have yielded favorable results by limiting the amount of cellular death (see, Shi, Z. W., et al., Frontiers in Pharmacology 9 (2018); Zhang, S., et al., Cell Death & Disease 8 (2017)). Although several RIPK3 kinase inhibitors have been created, they have not been extensively studied in vivo as RIPK1 kinase inhibitors therefor data is lacking in that regard, but it has been noted that the first identified RIPK3 kinase inhibitors induced RIPK3 dependent apoptosis (see, Mandal, P. et al. Molecular Cell 56, 481-495 (2014)). As for MLKL, it has had far fewer successful developed inhibitors with several having issues in specificity and only one successfully inhibiting necroptosis through MLKL inactivation by forming a covalent adduct to a surface exposed cysteine (see, Zhuang, C. L. & Chen, F. E. Journal of Medicinal Chemistry 63, 1490-1510 (2020)).

Currently, development of RIPK1 and RIPK3 inhibitors have focused on the inhibition of the protein kinase functions. In order to more fully expand the repertoire of available necroptosis inhibitors, experiments conducted during the course of developing embodiments for the present invention targeted protein-protein interactions within RIPK3 and MLKL in order to disrupt the last known activation step in necroptotic cell death. Such experiments demonstrated the identification of two distinct compounds obtained from in silico docking that will inhibit RIPK3 and MLKL binding through occupation of a binding pocket within the RIPK3 kinase domain

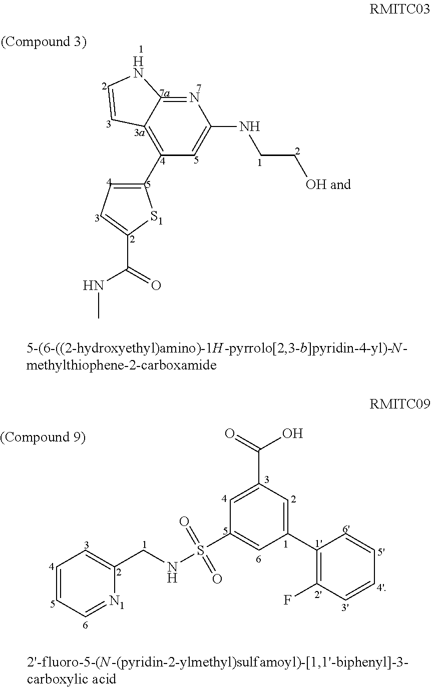

The compounds are referred to as RIPK3 MLKL Inhibitory Targeting Compounds (RMITC) herein. Indeed, such experiments showed that by targeting RIPK3, a subsequent reduction in MLKL activation and necroptotic cell death were observed.

Accordingly, the present invention relates generally to neurodegenerative diseases and conditions (e.g., Alzheimer's disease) characterized with aberrant RIPK1/RIPK3 binding, RIPK3/MLKL binding, necrosome formation, and/or necroptosis activation. This invention further relates to methods and compositions for treating such neurodegenerative diseases and conditions with pharmaceutical compositions comprising agents capable of inhibiting RIPK1/RIPK3 binding and/or RIPK3/MLKL binding.

In certain embodiments, the present invention provides a method of treating a mammal suffering from a neurodegenerative disorder comprising administering to the mammal a pharmaceutical composition comprising one or more agents capable of inhibiting RIPK1/RIPK3 binding and/or RIPK3/MLKL binding.

In some embodiments, the composition is capable of protecting neurons from necrosome formation and/or necroptosis activity.

In some embodiments, wherein the neurodegenerative disorder is selected from AD, Parkinson's disease, Huntington's disease, amyotrophic lateral sclerosis, motor neuron disease. In some embodiments, the AD is an early stage, prodromal phase of AD.

In some embodiments, the mammal is a human patient.

In some embodiments, the agent capable of inhibiting RIPK1/RIPK3 binding and/or RIPK3/MLKL binding is

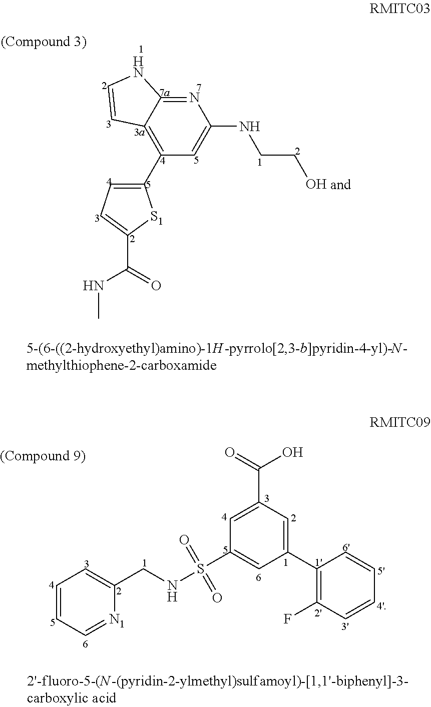

In certain embodiments, the present invention provides a method for preventing and/or inhibiting neuronal RIPK1/RIPK3 binding in a mammal in need thereof, the method comprising administering to the mammal a composition comprising one or more agents capable of inhibiting RIPK1/RIPK3 binding and/or RIPK3/MLKL binding.

In some embodiments, the composition is capable of protecting neurons from necrosome formation and/or necroptosis activity.

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

In some embodiments, the agent capable of inhibiting RIPK1/RIPK3 binding and/or RIPK3/MLKL binding is

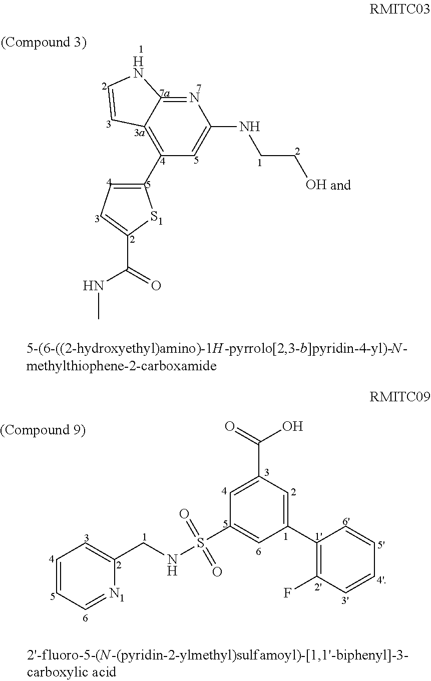

In certain embodiments, the present invention provides a method for preventing and/or inhibiting neuronal RIPK3/MLKL binding in a mammal in need thereof, the method comprising administering to the mammal a composition comprising one or more agents capable of inhibiting RIPK1/RIPK3 binding and/or RIPK3/MLKL binding.

In some embodiments, the composition is capable of protecting neurons from necrosome formation and/or necroptosis activity.

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

In some embodiments, the agent capable of inhibiting RIPK1/RIPK3 binding and/or RIPK3/MLKL binding is

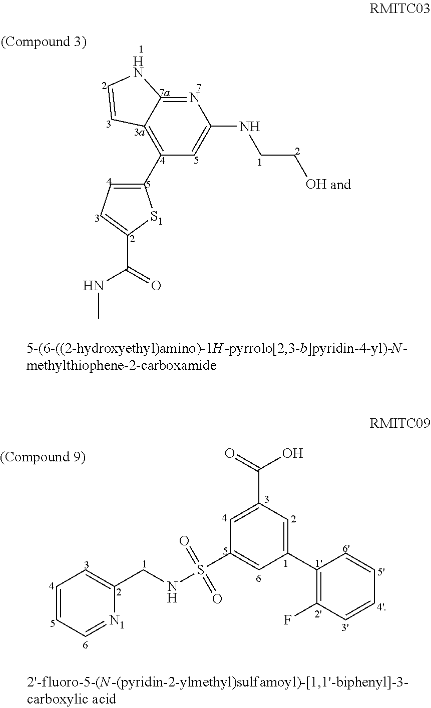

In certain embodiments, the present invention provides a method for preventing and/or inhibiting neuronal necrosome formation and/or necropotosis activity in a mammal in need thereof, the method comprising administering to the mammal a composition comprising one or more agents capable of inhibiting RIPK1/RIPK3 binding and/or RIPK3/MLKL binding.

In some embodiments, the mammal is suffering or at risk of suffering from a neurodegenerative disorder selected from AD, Parkinson's disease, Huntington's disease, amyotrophic lateral sclerosis, motor neuron disease.

In some embodiments, the mammal is a human patient.

In some embodiments, the agent capable of inhibiting RIPK1/RIPK3 binding and/or RIPK3/MLKL binding is

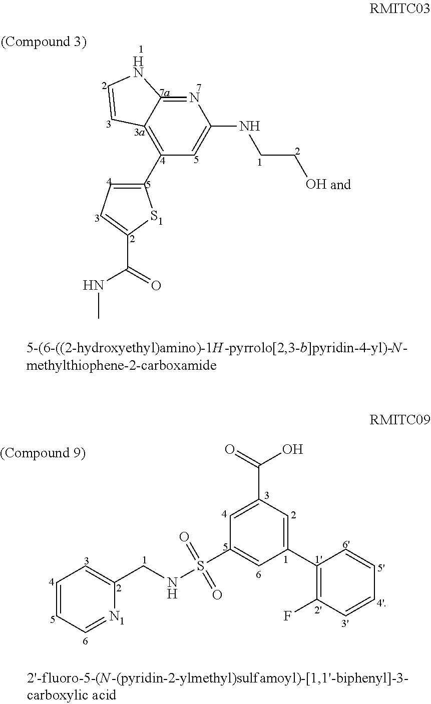

### BRIEF DESCRIPTION OF THE DRAWINGS

FIG. 1: A. The mouse RIPK3 (in red) and mouse MLKL pseudokinase domain (in green) crystal structure demonstrating the face to face contacts between the two domains. The non hydrolyzable ATP analog, AMP-PNP), is shown in sticks. The boxed area highlights the targeted interaction between RIPK3 and MLKL. B. Expanded view of targeted hydrophobic pocket within RIPK3 from boxed area in A. Phenylalanine (F373) from MLKL shown in stick in green. C. Surface view of RIPK3 demonstrating distance of pocket targeted (circled in green) from kinase active site which contains AMP-PNP shown in stick. D-E. In silico docking results of RMITC03 and RMITC09, respectively, within the targeted RIPK3 hydrophobic targeted. RIPK3 residues constituting the pocket are labelled and hydrogen bonds shown in yellow dashes. F. HT-29 cells were treated with 100 μM GSK'872 and compounds identified through the silico docking screen for 30 minutes prior to necroptosis induction for 7 hours with 25 ng/mL TNF-a, 20 μM Z-VAD428 fink, and 0.1 μM SM-164. HT-29 cells were stained with Annexin V and propidium iodide and analyzed by flow cytometry. Positively stained cell populations were categorized as necroptotic and compared to DMSO using the Holm-Sidak method with alpha of 0.05 to determine statistical significance with P value <0.005. Statistical significance is indicated by *. G. Chemical structures of positive necroptosis inhibitors, RMITC03 and RMITC09, identified in F.

FIG. 2: Compounds were named RMIT for RIPK3/MLKL interaction targeting compounds and numbered according to Glide scores (1 is highest, 13 is lowest) which approximates free energy binding of ligands. Compounds are commercially available from ChemBridge with the Hit2lead identification numbers indicated as well as chemical names and structures.

FIG. 3: HT-29 cells were stimulated to undergo necroptosis following 30 minute treatment with indicated compounds at 100 μM. Samples were collected and analyzed after 7 hours of necroptosis. With the exception of the cell only blank, cell stained annexin V (AnnV), and cell stained propidium iodide (PI), the remaining compound treated cells were induced for necroptosis. The gating strategy to select for populations within each compound treatment is demonstrated for each of the triplicate samples. The first population (first column within each triplicate group plotted FSC-Area vs SSCArea) was selected to obtain as much of the population that did not contain cellular debris. The second population (second column within each group plotted FSC-Area vs FSC-Height) was selected to obtain only a single t cell population. The last column separated the cell staining into AnnV−PI− (lower left quadrant), AnnV+PI− (Upper left quadrant), AnnV+PI+(Upper right quadrant), and AnnV−PI+(lower right quadrant). AnnV+PI−, AnnV+PI+, and AnnV−PI+ quadrants were categorized and plotted as necroptosis due to induction of necroptosis only.

FIG. 4: A. Representative western blot of CETSA assay indicates a decrease in RIPK3 solubility as temperature increases for the DMSO, 100 μM RMITC03, and 100 μM RMITC09 treated cells. B. Quantification of western blot band intensities were normalized to the highest (100%) and lowest (0%) signal. RIPK3 intensities for each treatment were plotted against the temperature point of incubation and a Boltzmann sigmoidal fit was applied. A clear shift in the thermal aggregation temperature (Tagg) is seen when compared to the DMSO sample. C. Increasing concentrations of RMITC03 and RMITC09 were incubated with HT-29 cells for 30 minutes prior to necroptosis induction (25 ng/mL TNF-a, 20 μM of Z-VAD-fmk, and 0.1 μM SM-164) for 6 hours. AlamarBlue™ Cell Viability Reagent was added for 1 hour before measurement. Data values were normalized to the DMSO treated sample and non-induced HT-29 cells.

FIG. 5: Size of molecular weight standards are indicated to the left of each blot and temperature point for incubation is indicated at the top. The band in each western blot corresponds to RIPK3. Triplicate CETSA assay western blot of DMSO (A), RMITC03 (B), and RMITC09 (C).

FIG. 6: HT-29 cells treated with 100 μM RMITC03, RMITC09, GSK'872, and Nec-1 as well as volume equivalent of DMSO. Compound treatments were incubated for 30 minutes prior to necroptosis induction with 25 ng/mL TNF-a, 20 μM of Z-VAD-fmk, and 0.1 μM SM-164, which is indicated by T/S/Z. The non-treated sample was not induced for necroptosis nor treated with any compound. Statistical significance is indicated by * for samples compared to DMSO with P value <0.005 using the Holm-Sidak method with alpha of 0.05. A. Representative western blot of RIPK1, RIPK3, and MLKL in HT-29 cells induced and non-induced for necroptosis under compound treatment B. Quantification of band intensities for RIP1, RIP3, and MLKL were normalized to the intensity of b-actin. No statistical significance was observed for RIPK1, RIPK3, and MLKL signal when compared to DMSO. C. Representative western blot of activated and phosphorylated RIPK1. RIPK3, and MLKL in HT-29 cells. D. As in B, band intensities were normalized to b-actin. When compared to DMSO, Nec-1, GSK'872, and the non-treated HT-29 cell had significant decrease in the amount of activated phospho-RIP1(Ser166). Phospho-RIP3(Ser227) only contained a significant decrease when GSK'872 treated cells were compared to DMSO. Phospho-MLKL(Ser358) had significant decrease at all treatments (RMITC03, RMITC09, GSK'872, and Nec-1) and non treated sample when compared to DMSO.

FIG. 7: Scheme of synthesis of A. RMITC03 and B. RMITC09 and their analogs. Proposed sites of modifications for the RMITC03 and RMITC09 analogs are also proposed.

### DETAILED DESCRIPTION

Alzheimer's disease (AD) is a progressive and degenerative brain disorder that is the leading cause of dementia, Epidemiological studies have indicated the prevalence rate for AD to be doubling every 5 years and expected to reach 114 million by 2050. Currently, AD is behind cancer and coronary heart disease as the most expensive disorders in the United States. There are currently no cures for Alzheimer's disease.

Despite many years of research and development, only four cholinesterase inhibitors and memantine have been approved by the FDA for the treatment of AD. These drugs only treat the symptoms associated with AD but do not alleviate the progressive nature of the disorder. Therefore, there is an urgent need for new therapeutics. Toward this end, experiments conducted during the course of developing embodiments for the present invention identified a novel target to alleviate some of the effects of AD. The novel target linked to AD and to other neurodegenerative diseases is a RIPK3/MLKL protein interface to inhibit necroptosis.

Recent reports have implicated necroptosis, a form of cell death, to be activated in human AD brains. The activation of necroptosis occurs through multiple protein-protein interactions beginning with RIPK1. RIPK3, and leading to MLKL phosphorylation. Activation of TNFα sets a rapid chain of intracellular signaling events that lead to RIPK11 activation. RIPK11 phosphorylates RIPK3, that then phosphorylates MLKL. Necroptosis is activated in postmortem human AD brains and in animal models of AD. Our preliminary data also show that RIPK1 inhibition significantly reduces neuronal loss in a mouse model of AD. However, targeting RIPK1 has been previously attempted and has not yielded fruitful drugs. It is likely because RIPK1 has other kinase functions outside of necroptosis.

Experiments conducted during the course of developing embodiments for the present invention targeted a binding pocket within RIPK3 known to be important for MLKL binding. Through in silico docking, such experiments identified two distinct compounds (RMITC03 and RMITC09) that bind RIPK3 and disrupt MLKL phosphorylation. It was shown that the resultant inhibition lead to less necroptotic cell death. These results not only demonstrate necroptosis may be ameliorated through targeted inhibition of sites of known protein-protein interactions but that the identified site within RIPK3 is highly amenable for further development and improvement of potential therapeutics against necroptosis.

Accordingly, the present invention relates generally to neurodegenerative diseases and conditions (e.g., Alzheimer's disease) characterized with aberrant RIPK1/RIPK3 binding, RIPK3/MLKL binding, necrosome formation, and/or necroptosis activation. This invention further relates to methods and compositions for treating such neurodegenerative diseases and conditions with pharmaceutical compositions comprising agents capable of inhibiting RIPK1/RIPK3 binding and/or RIPK3/MLKL binding.

An important aspect of the present invention is that the pharmaceutical compositions comprising agents capable of inhibiting RIPK1/RIPK3 binding and/or RIPK3/MLKL binding are useful in treating neurodegenerative diseases and conditions (e.g., Alzheimer's disease).

Some embodiments of the present invention provide methods for administering an effective amount of pharmaceutical compositions comprising agents capable of inhibiting RIPK1/RIPK3 binding and/or RIPK3/MLKL binding and at least one additional therapeutic agent (including, but not limited to, any pharmaceutical agent useful in treating neurodegenerative diseases and conditions (e.g., Alzheimer's disease).

In certain embodiments, the present invention provides a method of treating a mammal suffering from a neurodegenerative disorder comprising administering to the mammal a pharmaceutical composition comprising one or more agents capable of inhibiting RIPK1/RIPK3 binding and/or RIPK3/MLKL binding.

In certain embodiments, the present invention provides a method for preventing and/or inhibiting neuronal cell death in a mammal in need thereof, the method comprising administering to the mammal a composition comprising a pharmaceutical composition comprising one or more agents capable of inhibiting RIPK1/RIPK3 binding and/or RIPK3/MLKL binding.

In certain embodiments, the present invention provides a method for preventing and/or inhibiting RIPK1/RIPK3 binding in a mammal in need thereof, the method comprising administering to the mammal a pharmaceutical composition comprising one or more agents capable of inhibiting RIPK1/RIPK3 binding and/or RIPK3/MLKL binding.

In certain embodiments, the present invention provides a method for preventing and/or inhibiting RIPK3/MLKL binding in a mammal in need thereof, the method comprising administering to the mammal a pharmaceutical composition comprising one or more agents capable of inhibiting RIPK1/RIPK3 binding and/or RIPK3/MLKL binding.

In certain embodiments, the present invention provides a method for preventing and/or inhibiting necrosome formation in a mammal in need thereof, the method comprising administering to the mammal a pharmaceutical composition comprising one or more agents capable of inhibiting RIPK1/RIPK3 binding and/or RIPK3/MLKL binding.

In certain embodiments, the present invention provides a method for preventing and/or inhibiting necroptosis activity in a mammal in need thereof, the method comprising administering to the mammal a pharmaceutical composition comprising one or more agents capable of inhibiting RIPK1/RIPK3 binding and/or RIPK3/MLKL binding.

In some embodiments, the neurodegenerative disorder is selected from AD, Parkinson's disease, Huntington's disease, amyotrophic lateral sclerosis, motor neuron disease. In some embodiments, the AD is an early stage, prodromal phase of AD. In some embodiments, the neurodegenerative disorder is characterized with aberrant RIPK1/RIPK3 binding, RIPK3/MLKL binding, necrosome formation, and/or necroptosis activation

In some embodiments, the agent capable of inhibiting RIPK1/RIPK3 binding and/or RIPK3/MLKL binding is

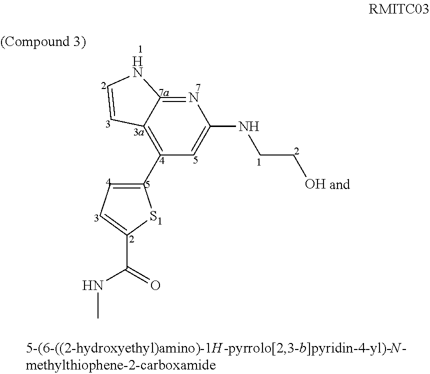

In some embodiments, the agent capable of inhibiting RIPK1/RIPK3 binding and/or RIPK3/MLKL binding is

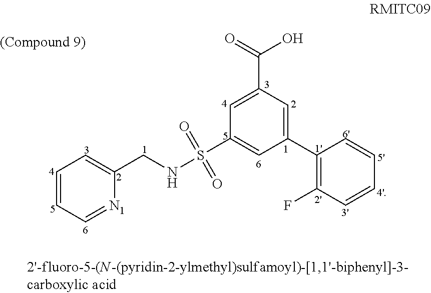

In some embodiments, the agent capable of inhibiting RIPK1/RIPK3 binding and/or RIPK3/MLKL binding is selected from:

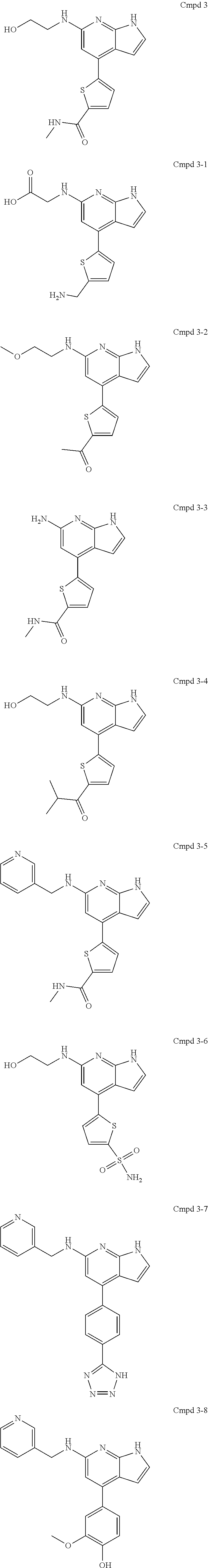

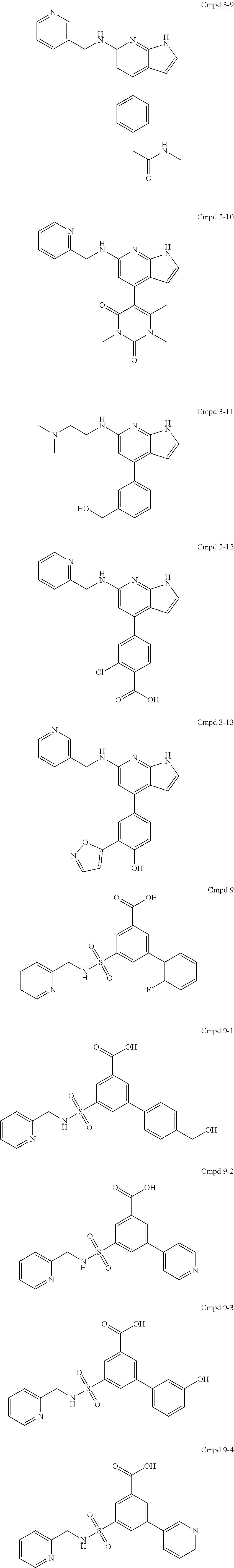

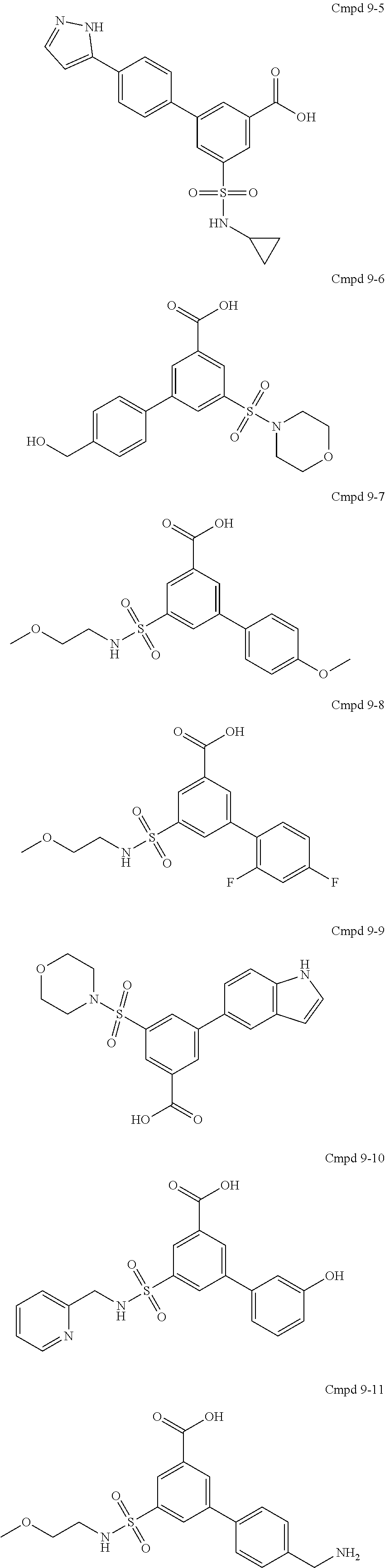

In certain embodiments, the present invention provides a method for preventing and/or inhibiting neuronal cell death in a subject suffering from a neurodegenerative disorder (e.g., AD (e.g., early stage, prodromal phase), Parkinson's disease, Huntington's disease, amyotrophic lateral sclerosis, motor neuron disease) comprising, consisting of, or consisting essentially of administering to the subject a therapeutically effective amount of an agent capable of inhibiting RIPK1/RIPK3 binding and/or RIPK3/MLKL binding.

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Treatment can include administration of an effective amount of one or more of an agents capable of inhibiting RIPK1/RIPK3 binding and/or RIPK3/MLKL binding.

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions capable of inhibiting RIPK1/RIPK3 binding and/or RIPK3/MLKL binding are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising an capable of inhibiting RIPK1/RIPK3 binding and/or RIPK3/MLKL binding and instructions for administering the agent to an animal (e.g., a human patient suffering from a neurodegenerative disorder (e.g., AD)). The kits may optionally contain other therapeutic agents.

### EXPERIMENTAL

The following examples are provided to demonstrate and further illustrate certain preferred embodiments of the present invention and are not to be construed as limiting the scope thereof.

### In Silico Docking of RIPK3/MLKL Interface.

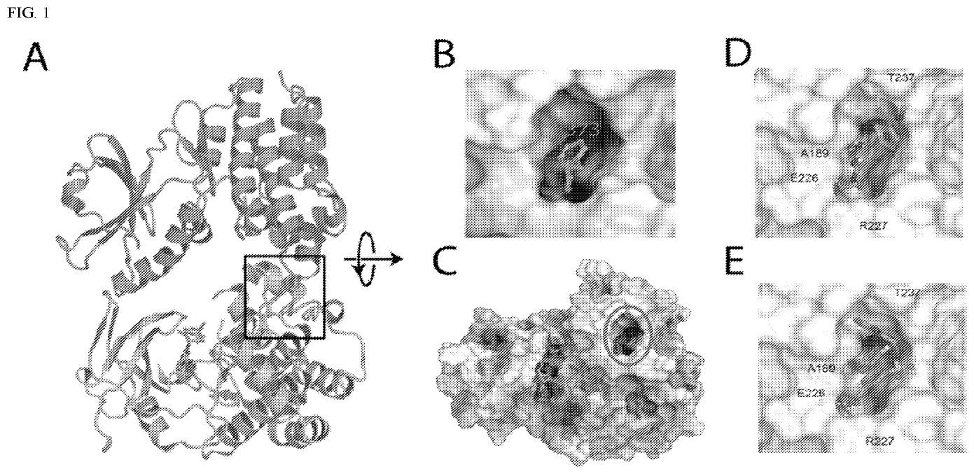

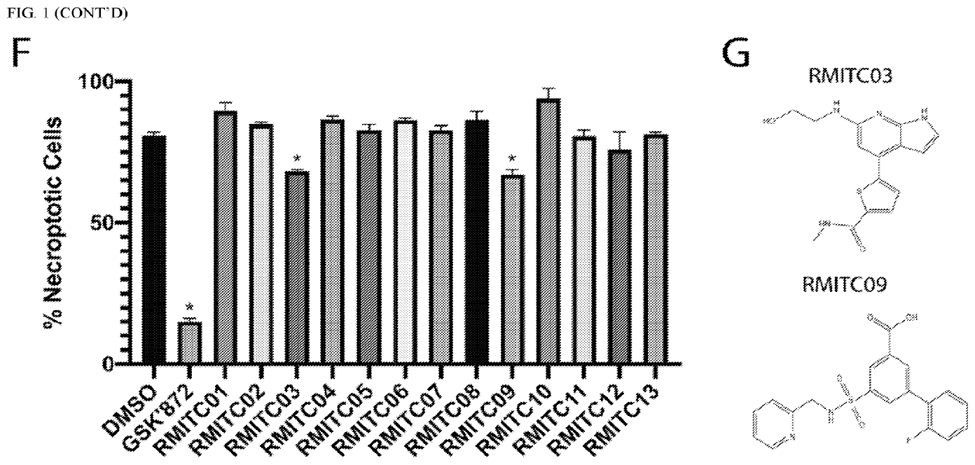

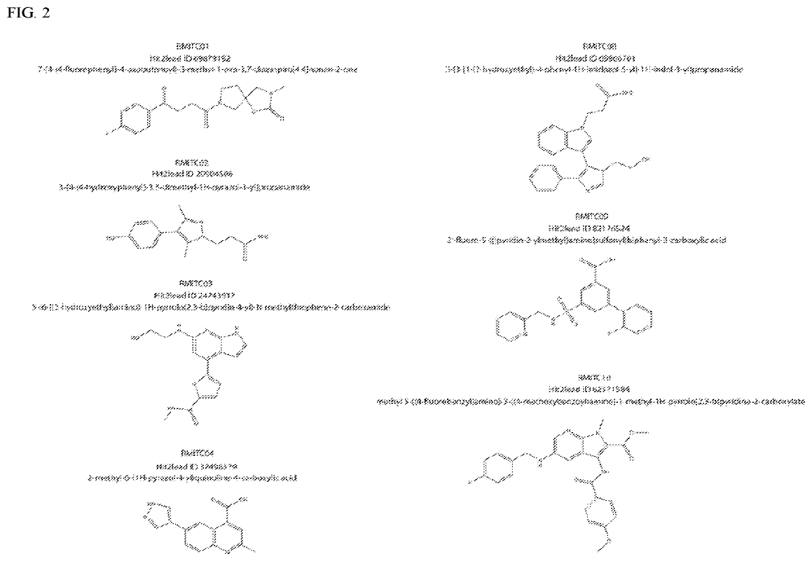

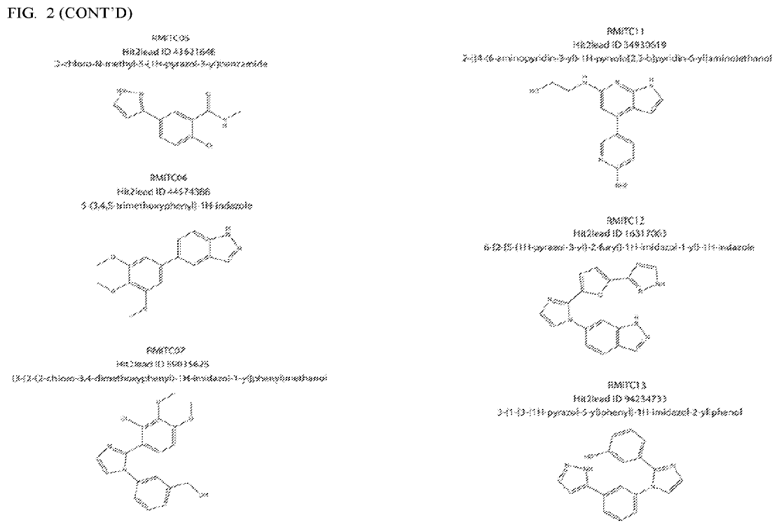

The co-crystal structure of the mouse RIPK3 kinase domain and mouse MLKL pseudokinase domain demonstrate large face to face protein-protein contacts with a phenylalanine residue (F373) from MLKL binding a pocket within RIPK3 (FIG. 1A-B). Mutations of_phenylalanine (F373) in mouse MLKL pseudokinase domain resulted in disruption of complex_formation with mouse RIPK3 kinase domain in vitro (see, Xie, T. et al. Cell Reports 5, 352 70-78 (2013)). The importance of this conserved interaction was demonstrated when expression of a mutant human MLKL (F386A) in MLKL^{-/-} human U937 cells failed to rescue necroptotic signaling (see, Petrie, E. J. et al. Cell Reports 28, 3309-+(2019)). Therefore, in order to identify possible compounds which might disrupt the interaction between RIPK3 and MKLKL, the conserved interaction between RIPK3 and MLKL was targeted through a chemical library screening 97 of 50,000 small molecules from Chembridge (FIG. 1C). The resultant screen identified 13 putative small compounds that might bind the targeted hydrophobic pocket (FIG. 2).

Targeting RIPK3/MLKL Interface Yields Compound that Inhibit Necroptosis.

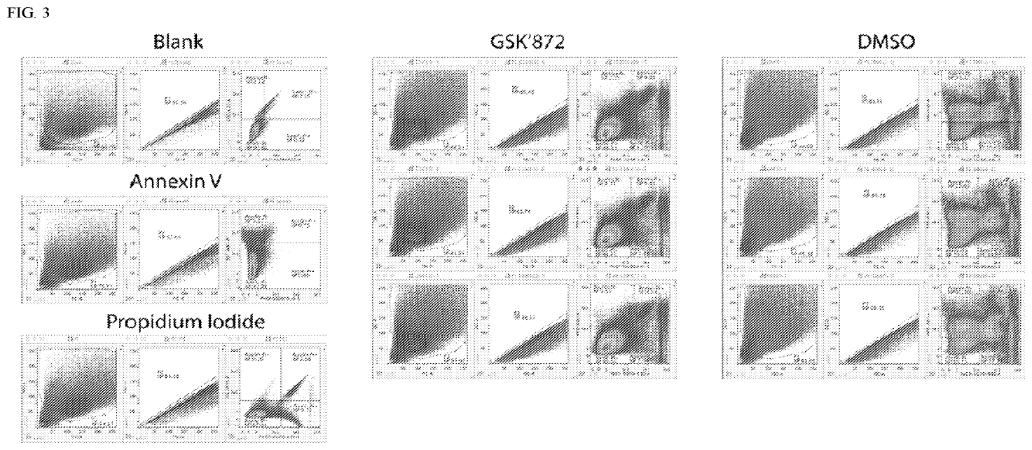

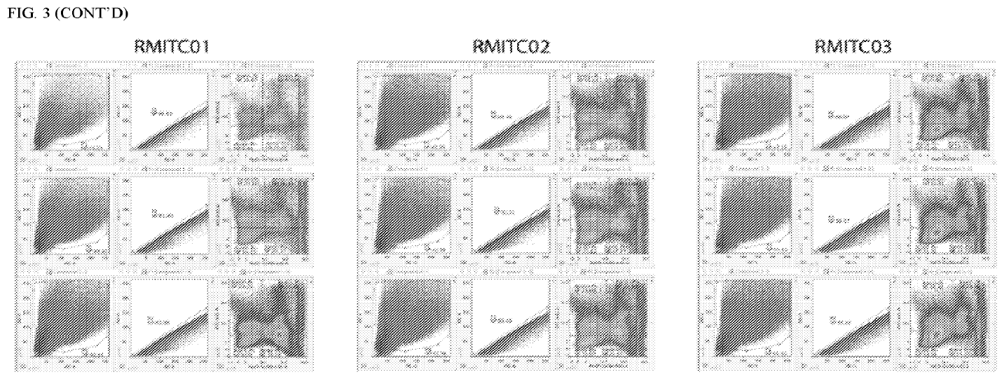

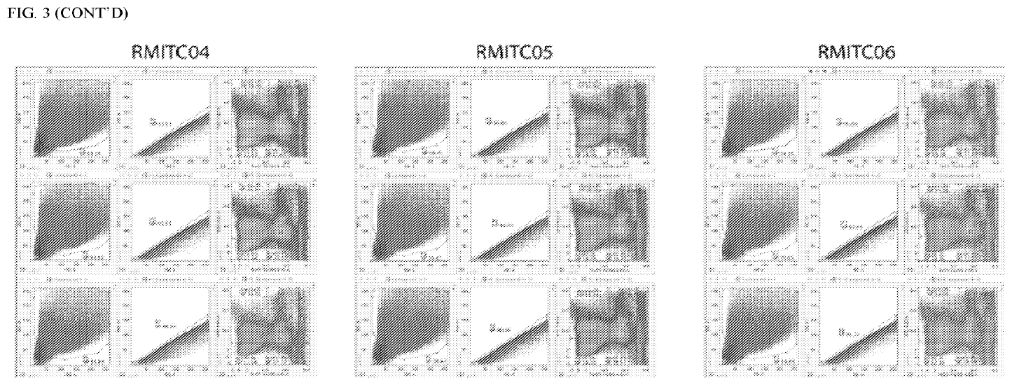

To ascertain which compounds had any inhibitory effect on necroptosis, HT-29 cells that readily undergo necroptosis specifically were utilized (see, Su, Z., et al., Cell Death and Differentiation 23, 748-756 (2016)). HT-29 cells were treated with 100 μM of putative identified compounds and a RIPK3 kinase inhibitor, GSK'872, for 30 minutes (min) prior to necroptosis induction with TNFa, SMAC mimetic (SM-164), and caspase inhibitor (Z-Vad fmk). Necroptosis was allowed to proceed for 7 hours (hrs) before Annexin V and propidium iodide staining and flow cytometry analysis (FIG. 3). Cells staining positive for either Annexin V or propidium iodide were quantified as necroptotic cells (FIG. 1F). Only three compounds had any inhibitory effect on necroptosis. GSK'872, as expected, had the largest inhibition of necroptosis while only two out of the 13 putative compounds identified through the docking screen had any inhibitory effect on necroptosis. The chemical structures of the two structurally distinct necroptotic inhibitors, RMITC03 and RMITC09, are indicated in FIG. 1G. Docking results from RMITC03 and RMITC09 indicate most interactions are driven through hydrophobic interactions with a few hydrogen bonds interacting with the backbone of the protein (FIG. 1D-E).

### CETSA Confirms Target Engagement of RIPK3 by RMITC09 and RMITC09.

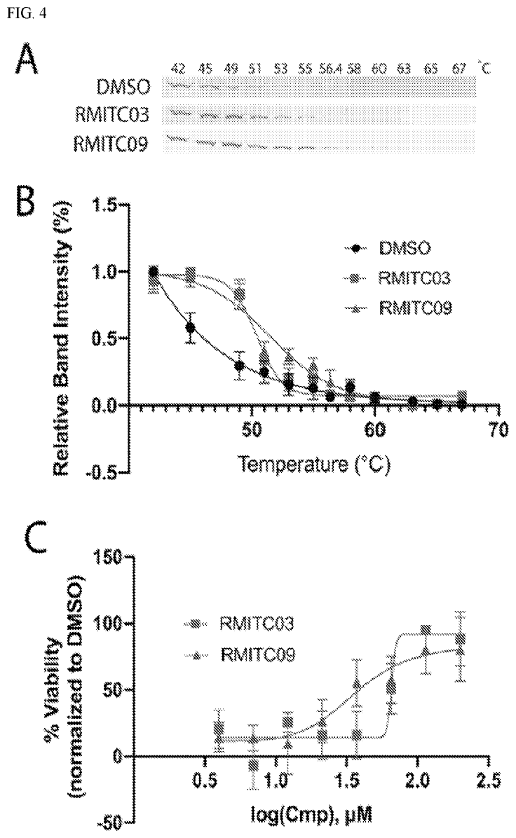

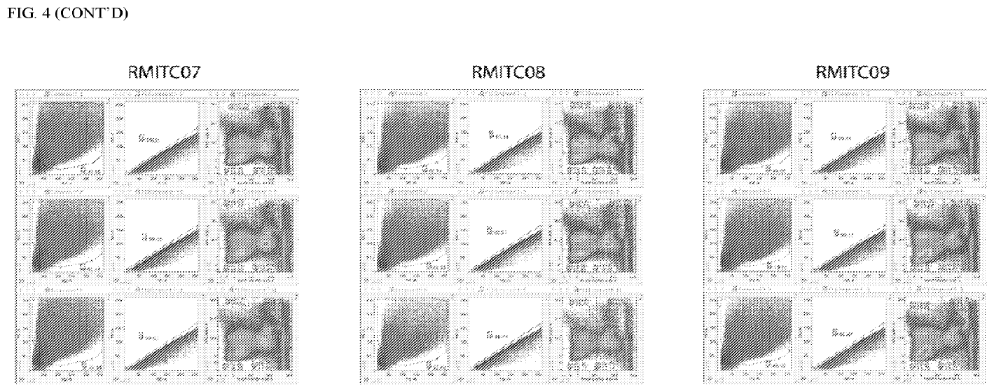

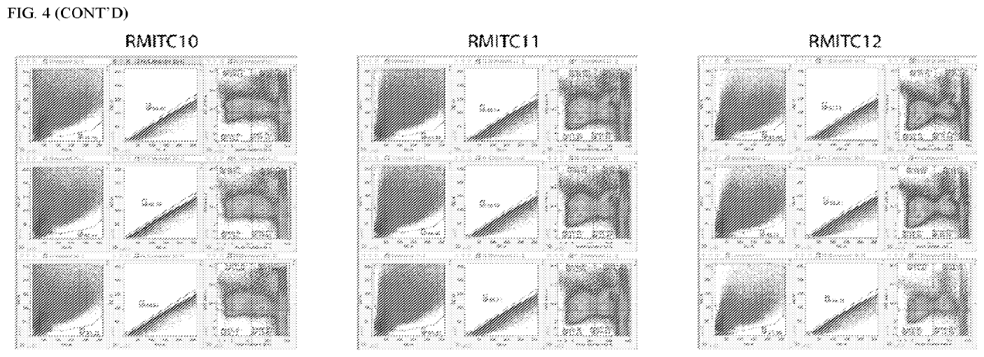

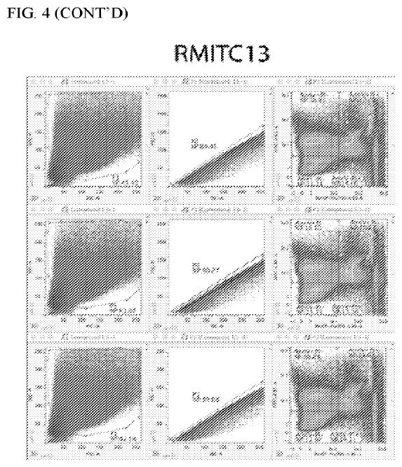

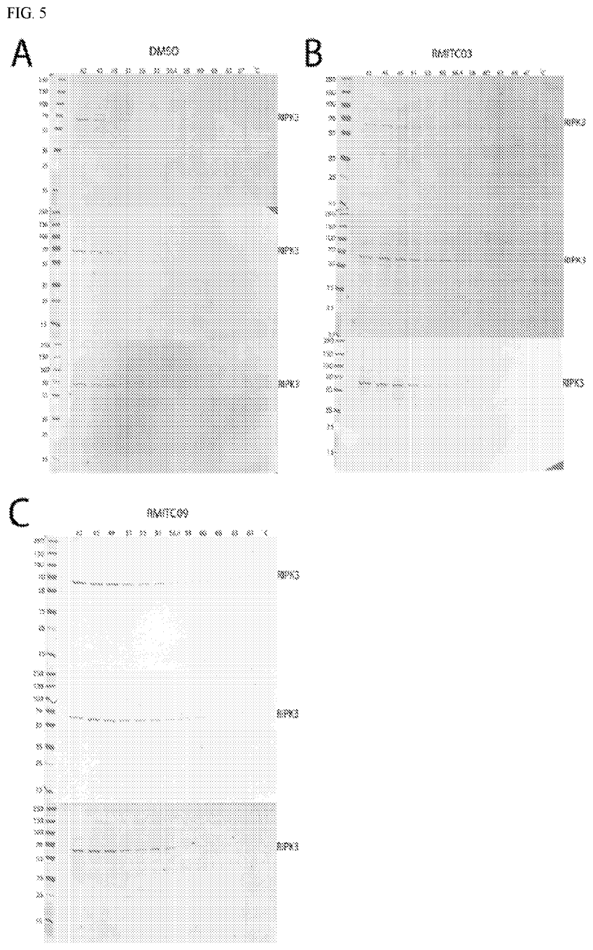

To confirm target engagement of RMITC03 and RMITC09 to RIPK3, experiments were conducted that utilized cellular_thermal shift assay (CETSA) to determine if RMITC03 and RMITC09 were able to bind to RIPK3_and stabilize the target at increasing temperatures in HT-29 cells. 100 μM of each compound was used in the CETSA assay to determine the thermal aggregation (Tagg) temperature, which is the point where 50% of the protein is unfolded and precipitated due to the increasing heat gradient. At increasing temperature points, RMITC03 and RMITC09 are stabilizing RIPK3 when compared to the DMSO control (FIG. 4A, FIG. 5). This is clearly observed by the shift in thermal melting curves of both RMITC03 and RMITC09 with Tagg values of 50.4 and 51.5° C., respectively (FIG. 4B). The Tagg for the DMSO control could not clearly be determined as it did not display a prominent sigmoidal pattern. In order to more accurately compare the effectivity of RMITC03 and RMITC09, a dilution series of each compound was performed and HT-29 cells were induced to undergo necroptosis for 6 hrs before cell viability measurements. Evaluating the effect of RMITC03 and RMITC09 in cells yielded an EC50 values of 65.5 and 36.3 μM, respectively (FIG. 4C).

RMITC03 and RMITC09 have No Effect on Total Level of Protein.

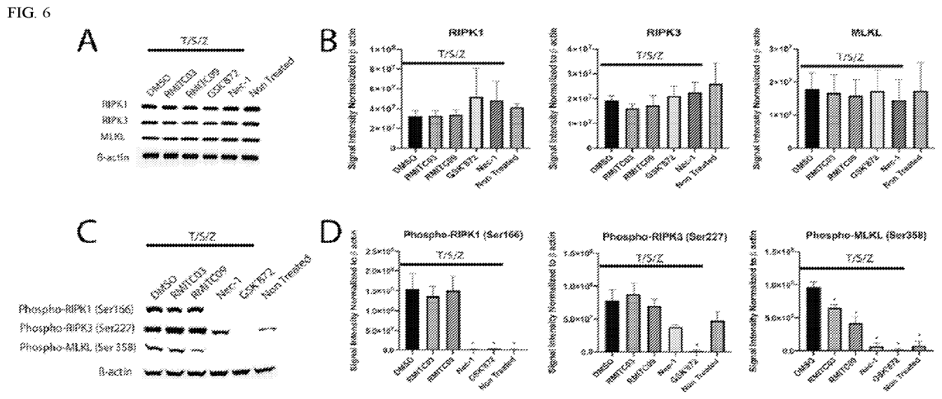

If RMITC03 and RMITC09 are disrupting the protein-protein interactions between RIPK3 and MLKL, then the effect of such inhibition should be observed in the activation of MLKL. To test this, HT-29 cells were treated with 100 μM of each compound for 30 min before necroptosis induction. Cells were harvested after 3 hrs with RIPA buffer in order to determine amount of inhibition at each stage of the necroptotic signaling molecules. When comparing the amounts of RIPK1, RIPK3, and MLKL in DMSO treated necroptosis activated cells (T/S/Z) against non necroptosis activated cells (Non-treated samples), there is no statistically significant difference in the amounts of these proteins (FIG. 6A-B). Similarly, 100 μM inhibitor treatment to necroptosis activated cells (RMITC03, RMITC09, GSK'872, and Nec-1) resulted in no significant difference in RIPK1, RIPK3, or MLKL (FIG. 6A-B) when compared to DMSO.

RMITC03 and RMITC09 have an Effect on MLKL Phosphorylation with No Effect on RIPK1 or RIPK3.

However, when quantifying the phosphorylated activated versions of RIPK1, RIPK3, and_MLKL, differences were observed for each protein under different inhibitor treatments (FIG. 6C-D). Autophosphorylation of RIPK1 at Ser166 facilitates RIPK1 kinase activity to induce cell_death (see, Meng, H. Y. et al. Proceedings of the National Academy of Sciences of the United States of America 115, E2001-E2009 (2018)). As such, phosphorylation at Ser166 was used to detect activation of necroptosis. Treatments with Nec-1, a known RIPK1 kinase inhibitor, and GSK'872, a RIPK3 kinase inhibitor, resulted in no observable phosphorylated RIPK11 signal. Similarly, in the non-necroptosis activated on treated sample, there is also no phosphorylated RIPK1 signal. When comparing the DMSO sample to RMITC03 and RMITC09 treated samples, there is a large amount of activated phosphorylated RIPK1 with no difference between the DMSO control and RMITC03 and RMITC09 compounds.

As previously reported, RIPK3 activation from necroptotic stimulus can be readily and accurately measured by phosphorylation at Ser227 (see, Chen, W. Z. et al. Journal of Biological Chemistry 288, 16247-16261 (2013)). Inhibition of activated phosphorylated RIPK3 at Ser227 was only seen when GSK'872 was used in the treatment of necroptosis activated cells. When comparing the DMSO treated cells that were induced to undergo necroptosis against RMITC03 and RMITC09, there was a difference observed in the amount of activated phosphorylated RIPK3 (FIG. 6C-D). Although not statistically significant, Nec-1 treatment did not completely inhibit activation of phosphorylated RIPK3 but its activated RIPK3 levels were similar to the non-treated control which was not necroptosis induced.

RIPK3 is known to activate MLKL through phosphorylation at Ser358 for necroptotic cell death (see, Sun, L. M. et al. Cell 148, 213-227 (2012)). Therefore, the amount of phosphorylated MLKL at Ser358 was used to determine if any inhibition of MLKL activation occurred. The amount of phosphorylated MLKL was significantly less for all samples treated with compounds and inhibitors when compared to the DMSO treated sample. The lowest levels of phosphorylated MLKL came from Nec-1 and GSK'872 treated samples as well as the non-treated control (FIG. 6C-D). RMITC03 and RMITC09 also had diminished levels of phosphorylated MLKL in comparison to DMSO, with compound RMITC09 showing improved diminishment in phosphorylated of MLKL over RMITC03.

### DISCUSSION

Although necroptosis is involved in cellular homeostasis and as part of the immune system, mounting evidence has indicated that several inflammatory diseases and neurodegenerative diseases are associated with necroptosis. Inhibition of key proteins in necroptosis activation have yielded positive results in many different disease models. To date, the majority of necroptosis inhibitors have been focused on the development of kinase inhibitors against RIPK1 and RIPK3 with several RIPK1 kinase inhibitors entering clinical trials (see, Sheridan, C. Biotechnology 37, 111-113 (2019)).

Experiments described herein identified a pocket within RIPK3 that has the potential to be used a target for inhibition of protein-protein interactions between RIPK3 and MLKL. Unexpectedly, GSK'872 inhibited activation of phospho-RIPK1 likely due to high concentration of GSK'872 used in our studies for direct comparison with RMITC inhibitors. Other studies have used it at 180 μM, a concentration that is only 10-fold less than the concentration used here (see, Yang, X. S. et al. Scientific Reports 7 (2017); Cekay, M. J. et al. Cancer Letters 410, 228-237 (2017)). However, GSK'872 has an IC50 of 1.8 nM and using 100 μM of GSK'872, this may cause off-target inhibition of RIPK1 (see, Mandal, P. et al. Molecular Cell 56, 481-495 (2014)). Although Nec-1 working concentrations have been used in ranges from 5-300 μM under different cell types and necroptosis inducing conditions, several off-target kinase effects of Nec-1 have been identified (see, Vandenabeele, P., et al., Cell Death and Differentiation 20, 185-187 (2013); Biton, S. & Ashkenazi, A. Cell 145, 92-103 (2011); Nehs, M. A. et al. Surgery 150, 1032-1038 (2011); Zheng, H. W., Chen, J. & Sha, S. H. et al., Cell Death & Disease 5 (2014); Wu, J. R. et al. Neural Regeneration Research 10, 1120-1124 (2015)). Therefore, necroptosis inhibition through the identified pocket within RIPK3 could provide an alternative to develop strategies for a structure guided design to improve specificities of novel necroptosis inhibitors to minimize off-target effects observed in the development of kinase inhibitors (see, Lin, A. et al. Science Translational Medicine 11 (2019); Hantschel, O. Acs Chemical Biology 10, 234-245 (2015)). The two structurally distinct chemotype compounds, RMITC03 and RMITC09, demonstrated an ability to inhibit necroptosis at the last step of activation via MLKL phosphorylation before necroptotic cell death. Although the compounds are relatively weak in their inhibitory effect, it should be noted that these compounds are the first identified compounds to be able to inhibit necroptosis through protein-protein interactions. As experiments continue to build and improve upon these initial hits, we have already obtained improved necroptosis inhibitors. Therefore, these compounds should be taken as proof in principle of the ability to inhibit necroptosis through targeting the conserved interaction between the RIPK3 hydrophobic pocket and the phenylalanine of MLKL.

As more studies regarding necroptosis and RIPK3-MLKL interactions are elucidated through structural studies, it will become clearer as to how conformational changes are facilitating recruitment and activation of the necroptotic signaling molecules. This in turn, may lead to further understandings of this signaling pathway in order to identify novel targets for therapeutic development.

### Experimental Procedures

### Schrödinger in Silico Screen

The Schrödinger suite of programs were utilized to perform docking on mouse RIPK3 kinase domain from the X-ray crystal structure of mouse RIPK3 kinase domain and mouse MLKL_pseudokinase domain (PDB: 4M69). The protein structure were processed using the Protein preparation wizard to remove any waters and mouse MLKL pseudokinase domain (see, Sastry, G. M., et al., Journal of Computer-Aided Molecular Design 27, 221-234 (2013)), A 10 Å³ grid was placed around the mouse RIPK3 domain hydrophobic pocket that centered on residues Thr237, Ala189, Glu226, and Arg227. The Glide program within Schrödinger was used to screen the DIVERSet-CL library which contains 50,000 small molecules available from ChemBridge Corp. The resultant 13 compounds were ranked by Glide score, which is an approximation of ligand binding free energy (see, Friesner, R. A. et al. Journal of Medicinal Chemistry 49, 6177-6196 (2006)).

### Compounds

Compounds from in silico docking (FIG. 2) were purchased from ChemBridge. GSK'872 and Nec-1 were purchased from MedChemExpress HY-101872 and AdooQ Bioscience_A11973, respectively. All compounds and inhibitors were solubilized in DMSO (Corning 25-950-218 CQC).

### Necroptosis Flow Assay

HT-29 cells were grown in DMEM (Corning 10-013-CV) with 10% FBS (HyClone SH3091003)_and incubated at 37° C. and 5% CO2. HT-29 cells were incubated with 100 μM of each of the 13 compounds identified in the docking (FIG. 2) and GSK'872 for 30 minutes prior_to necroptosis induction. Necroptosis was induced with 25 ng/mL TNF-a (Prospec CYT-223), 20_μM Z-VAD-fmk (AdooQ Bioscience A12373), and 0.1 μM SM-164 (AdooQ Bioscience A15800) for 7 hours in an incubator at 37° C. and 5% CO2. HT-29 cells were trypsinized (Corning 25-053-CI) and washed with DPBS (Corning 21-031-CV) (see, Bundscherer, A. et al. Anticancer Research 33, 3201-3204 (2013)). Cells were processed and stained with Annexin V and propidium iodide as described by manufacturer's protocol in the eBioscience™ Annexin V-FITC Apoptosis Detection Kit (Invitrogen BMS500FI-500). Flow cytometry was performed with MACSQuant® Analyzer 10 and data was processed with Flowlogic™ flow cytometry analysis software. Since necroptosis was specifically induced, all populations staining positive for Annexin-V and/or propidium iodide were categorized as necroptotic (see, Wallberg, F., Tenev, T. & Meier, P. Analysis of Apoptosis and Necroptosis by Fluorescence-Activated Cell Sorting. Cold Spring Harbor protocols 2016 405 (2016).

### Cell Viability

HT-29 cells were grown as previously described and plated at 50,000 cells/well in a black clear bottom 96 well plate (Greiner 655090). After overnight incubation, cells were treated with varying concentrations of RMITC03 and RMITC09 for 30 minutes prior to necroptosis induction as described above. Necroptosis was allowed to proceed for 6 hours before addition of alamarBlue™ Cell Viability Reagent (Invitrogen DAL1025) for 1 hour at a final concentration of 1×. All incubation times were carried out in an incubator at 37° C. and 5% CO2. Measurements were performed with a BioTek® Cytation 5 instrument with the following setting: 560 nm for excitation and 590 nm emission. Data was normalized to the non necroptosis induced HT-29 cells (Cell only) and necroptosis induced HT-29 cells with DMSO using the following equation:

$$\% Viability = \frac{Compound - DMSO}{Cell only - DMSO} \times 100 \%$$

### CETSA

To measure target engagement from the compounds, cellular thermal shift assay (CETSA) was_employed (see, Ishii. T. et al. Scientific Reports 7 (2017); Jafari, R. et al. Nature Protocols 9, 2100-2122 (2014); Molina, D. M. et al. Science 341, 84-87 (2013)). Cells were trypsinized and washed with DPBS before resuspending in DMEM with 10% FBS and 100 μM of RMITC03 and RMITC09. 30 million cells were resuspended in 10 ml of the media with compound in a 15 mL tube. Cells and compounds were incubated for 1 hour at 37° C. and 5% CO2 with gentle mixing by inversion every 10 min. Cells were centrifuged at 200×g for 5 min at room temperature and washed with DPBS containing 100 μM of RMITC03 and RMITC09. A wash with DPBS with 100 μM of RMITC03 and RMITC09 and protease inhibitor cocktail set III (Millipore 539134) was repeated and cells were aliquoted at 3 million cells per PCR tube at 100 μL. Samples in PCR tubes were placed Biorad T100 Thermal Cycler and incubated at increasing temperatures for 3 min. PCR samples were immediately frozen in liquid nitrogen and thawed at 25° C. on the Biorad T100 Thermal Cycler. Freezing and thawing was repeated two more times before centrifugation at 20000×g for 10 min at 4° C. 70μL of supernatant was removed and added to 30 μL of NuPAGE LDS sample buffer dye 4×(Invitrogen NP0008) with 0.1 M DTT. Samples were heated at 90° C. for 10 min before running samples on SDS-PAGE (Invitrogen XP04205BOX). Gels were transferred to PVDF membrane using iBlot™ 2 system (Invitrogen). Membranes were blocked for 1 hour at room temperature with 5% milk (Omniblock AB10109) in PBS, and incubated overnight with RIP3 antibody (B-2) Alexa Fluor 488 (Santa Cruz Biotechnology sc-374639 AF488) at 1:500 in PBS-T with 5% BSA at 4° C. on a rocker. Membranes were developed using the Chemidoc™ MP imaging system under the Alexa 488 settings. Western blots were quantified using ImageLab™ software. The data was normalized to the lowest (0%) and highest (100%) value for each western blot prior to plotting and fitting to Boltzmann sigmoidal equation using Prism 8 software.

### Western Blots

HT-29 cells were grown as described above and plated on a 6 well plate. After incubating overnight, cells were incubated with 100 μM of noted compounds in FIG. 3 for 30 min._Necroptosis was induced in the cells for 3 hours, as described in Necroptosis flow assay section, before harvesting with 75 μL RIPA buffer system (Santa Cruz sc-24948) containing 2 mM PMSF, 1 mM Sodium Orthovanadate, and 1% final volume of protease inhibitor. Samples were_centrifuged for 10 min at 20000×g and 4° C. The supernatant was mixed with 25 μL of NuPAGE_LDS sample buffer dye 4× containing 0.1 M DTT and heated for 10 min at 90° C. SDS-PAGE_separation and membrane transfer were performed as described under the CETSA protocol. The_following primary antibodies were used at a 1:1000 dilution factor: Phospho-RIPK1 (Ser166) (Cell Signaling Technology #65746S), RIPK1 (Cell Signaling Technology #3493S), Phospho-RIPK3 (Ser227) (Cell Signaling Technology #93654S), RIPK3 (Cell Signaling Technology #13526S), Phospho-MLKL (Ser358) (Cell Signaling Technology #91689S), and MLKL (Cell Signaling Technology #14993S). β-actin antibody (C-4) Alexa Fluor 790 (Santa Cruz Biotechnology sc-47778 AF790) was also used as a primary antibody at a dilution of 1:500 in PBS-T with 5% BSA.

The secondary Anti-rabbit IgG, HRP-linked antibody (Cell Signaling Technology #7074S) was_used at a dilution of 1:1500 in PBS-T with 5% BSA. The membrane was incubated with secondary antibody for 1 hour at room temperature while gently rocking before development with Pierce™ ECL Western Blotting Substrate (Thermo Scientific 32109). Membranes were developed using the Chemidoc™ MP imaging system under the Chemilumiscence and Alexa 790 settings. Data_were quantified with ImageLab™ software. Antibody signals were normalized to β-actin and_plotted with Prism 8 software.

### Statistical Analysis

Experiments were conducted in triplicates and statistical significance was indicated where noted with * when compared to the DMSO sample. Statistical significance was determined with P value <0.005 using the Holm-Sidak method with alpha of 0.05.

### Synthesis of Additional Compounds

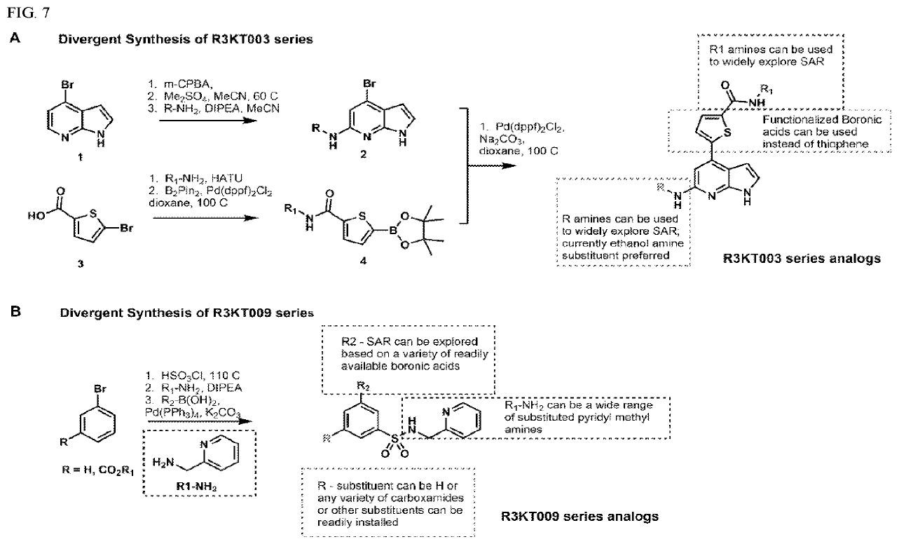

In addition to the RMITC03 and RMITC09 analogues, more analogues of RMITC03 and RMITC09 will be designed to systemically elucidate the SAR and improve biological and pharmacological properties (FIGS. 7A & 7B). The design is guided by computer modeling, drug design principle, ligand-target binding information, NMR and synthetic feasibility. Analysis of the structure of the R3KT003 reveals that the thiophene and the flexible aminol are possible place for modification (FIG. 7B). As bioisostere, commonly used 5- and 6-membered heteroaromatics will be incorporated into the structure. In addition, the linear aminol with different lengths and more rigid cyclic structure will be designed and synthesized for SAR studies. The rigid cyclics may improve potency and specificity. With respect to R3KT009 analogs, a computational design has provided 4 compounds, which represent an important guideline to address critical SAR. When these compounds are synthesized and tested, there will have a clearer SAR picture. In addition to these efforts, its analogs will be designed with the emphasis on the understanding of the substituent pattern effect including replacing F by Cl, OH, SH, Br, NH₂ and o-, m- and p-positions. Furthermore, other heteroaromatic bioisosteres such as pyridine, pyrrole, furan, thiophene, etc. will be probed.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating a mammal suffering from a neurodegenerative disorder comprising administering to the mammal a pharmaceutical composition comprising one or more agents capable of inhibiting RIPK1/RIPK3 binding and/or RIPK3/MLKL binding.

2. The method of claim 1, wherein the composition is capable of protecting neurons from necrosome formation and/or necroptosis activity.

3. The method of claim 1, wherein the neurodegenerative disorder is selected from AD, Parkinson's disease, Huntington's disease, amyotrophic lateral sclerosis, motor neuron disease.

4. The method of claim 3, wherein the AD is an early stage, prodromal phase of AD.

5. The method of claim 1, wherein the mammal is a human patient.

6. The method of claim 1, wherein the agent capable of inhibiting RIPK1/RIPK3 binding and/or RIPK3/MLKL binding is

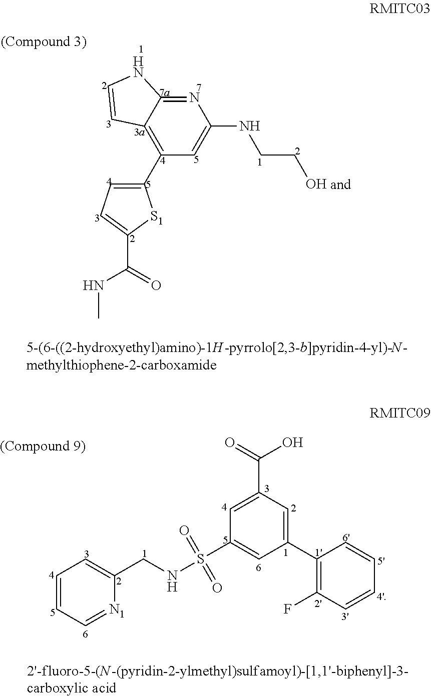

7. A method for preventing and/or inhibiting neuronal RIPK1/RIPK3 binding in a mammal in need thereof, the method comprising administering to the mammal a composition comprising one or more agents capable of inhibiting RIPK1/RIPK3 binding and/or RIPK3/MLKL binding.

8. The method of claim 7, wherein the composition is capable of protecting neurons from necrosome formation and/or necroptosis activity.

9. The method of claim 7, wherein the mammal is suffering or at risk of suffering from a neurodegenerative disorder selected from AD, Parkinson's disease, Huntington's disease, amyotrophic lateral sclerosis, motor neuron disease.

10. The method of claim 7, wherein the mammal is a human patient.

11. The method of claim 7, wherein the agent capable of inhibiting RIPK1/RIPK3 binding and/or RIPK3/MLKL binding is

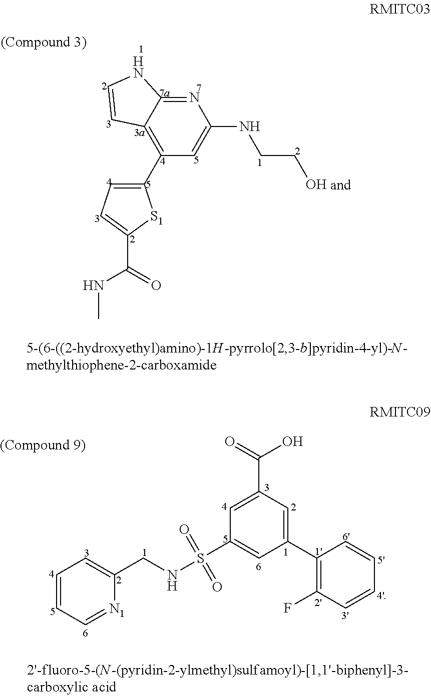

12. A method for preventing and/or inhibiting neuronal RIPK3/MLKL binding in a mammal in need thereof, the method comprising administering to the mammal a composition comprising one or more agents capable of inhibiting RIPK1/RIPK3 binding and/or RIPK3/MLKL binding.

13. The method of claim 12, wherein the composition is capable of protecting neurons from necrosome formation and/or necroptosis activity.

14. The method of claim 13, wherein the mammal is suffering or at risk of suffering from a neurodegenerative disorder selected from AD, Parkinson's disease, Huntington's disease, amyotrophic lateral sclerosis, motor neuron disease.

15. The method of claim 12, wherein the mammal is a human patient.

16. The method of claim 12, wherein the agent capable of inhibiting RIPK1/RIPK3 binding and/or RIPK3/MLKL binding is

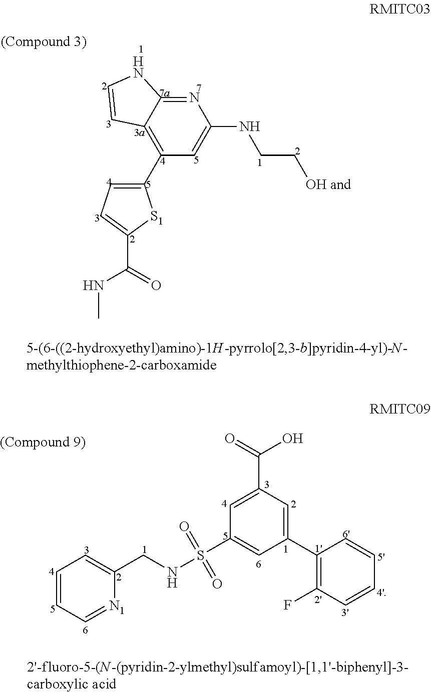

17. A method for preventing and/or inhibiting neuronal necrosome formation and/or necropotosis activity in a mammal in need thereof, the method comprising administering to the mammal a composition comprising one or more agents capable of inhibiting RIPK1/RIPK3 binding and/or RIPK3/MLKL binding.

18. The method of claim 17, wherein the mammal is suffering or at risk of suffering from a neurodegenerative disorder selected from AD, Parkinson's disease, Huntington's disease, amyotrophic lateral sclerosis, motor neuron disease.

19. The method of claim 17, wherein the mammal is a human patient.

20. The method of claim 17, wherein the agent capable of inhibiting RIPK1/RIPK3 binding and/or RIPK3/MLKL binding is [chemical structure]
